(19) United States
(12) Patent Application Publication
(10) Pub. No.: US 2021/0267822 A1
(43) Pub. Date: Sep. 2, 2021

(54) Elastic Super Absorbent Polymer Sheet

(71) Applicant: 3M INNOVATIVE PROPERTIES COMPANY, St. Paul, MN (US)

(72) Inventor: Wataru Nakamura, Tokyo (JP)

(21) Appl. No.: 17/250,220

(22) PCT Filed: Jun. 26, 2019
(86) PCT No.: PCT/IB2019/055414

§ 371 Date: Dec. 17, 2020

(30) Foreign Application Priority Data
Jun. 29, 2018 (JP) 2018-125135

(51) Int. Cl. A61F 13/532; A61F 13/536; A61F 13/15

(52) U.S. Cl. CPC A61F 13/5323; A61F 13/536; A61F 2013/53062; A61F 2013/15869; A61F 2013/15878; A61F 13/15577

(57) ABSTRACT

An absorbent body having elasticity in a plurality of directions is provided. The absorbent body according to an embodiment of the present invention includes a first elastic member, a second elastic member, and an absorbing member laminated between the first elastic member and the second elastic member, wherein the first elastic member forms, together with the second elastic member, a plurality of housing parts, and the plurality of housing parts are delimited from each other by a first delimiting part along a first direction and a second delimiting part along a second direction intersecting the first direction, and house the absorbing member.

### TECHNICAL FIELD

The present invention relates to an absorbent body, an absorbent article, and a method of manufacturing the absorbent body.

### BACKGROUND

An absorbent body capable of absorbing liquid is used in an absorbent article such as a disposable diaper and a sanitary napkin. JP 2009-297146 A discloses an absorbent structure used for an absorbent article, and in this absorbent structure, an absorbent is disposed interposed between a first sheet and a second sheet. In addition, this absorbent structure includes a region in which the absorbent is disposed (absorbent arrangement region) and a region in which no absorbent is disposed (absorbent non-arrangement region). The absorbent arrangement region and the absorbent non-arrangement region both extend in one direction and form a plurality of rows. In addition, in the absorbent arrangement region, the absorbent is fixed by an adhesive, and in the absorbent non-arrangement region, the sheets are fixed to each other by an adhesive.

### SUMMARY OF INVENTION

An absorbent body is required to follow various postures of a person wearing an absorbent article and to have elasticity reducing a gap with a skin of the wearer and enabling absorbing urine and the like without leakage of the urine and the like. Since the absorbent body described above fixes an absorbent by an adhesive, sufficient elasticity may not be obtained. In addition, such elasticity is required to be provided not only in one direction but also in a plurality of directions.

An absorbent body according to an embodiment of the present invention includes a first elastic member, a second elastic member, and an absorbing member laminated between the first elastic member and the second elastic member, and in the absorbent body, the first elastic member forms, together with the second elastic member, a plurality of housing parts, and the plurality of housing parts are delimited from each other by a first delimiting part along a first direction and a second delimiting part along a second direction intersecting the first direction, and house the absorbing member. An absorbent body having elasticity in a plurality of directions can be provided. An absorbent article including the absorbent body can be provided. In addition, a method of manufacturing the absorbent body having elasticity in a plurality of directions can be provided.

### BRIEF DESCRIPTION OF DRAWINGS

FIG. 1 is a perspective view schematically illustrating an absorbent body according to an embodiment.

FIG. 2 is a plan view schematically illustrating the absorbent body according to the embodiment.

FIGS. 3A and 3B are cross-sectional views schematically illustrating the absorbent body according to the embodiment.

FIGS. 4A and 4B are cross-sectional views schematically illustrating the absorbent body according to the embodiment.

FIGS. 5A and 5B are cross-sectional views schematically illustrating the absorbent body according to the embodiment.

FIG. 6A is a plan view schematically illustrating an absorbent body according to a comparative example, and FIG. 6B is a cross-sectional view schematically illustrating the absorbent body according to the comparative example.

FIGS. 7A and 7B are cross-sectional views schematically illustrating the absorbent body according to the comparative example.

FIG. 8 is a view illustrating an absorbent article including an absorbent body.

FIG. 9 is a view schematically illustrating an absorbent body manufacturing apparatus.

FIG. 10 is a flow chart illustrating a method of manufacturing an absorbent body according to an embodiment.

FIGS. 11A, 11B, and 11C are views illustrating products at steps in the method of manufacturing an absorbent body, respectively.

### DETAILED DESCRIPTION

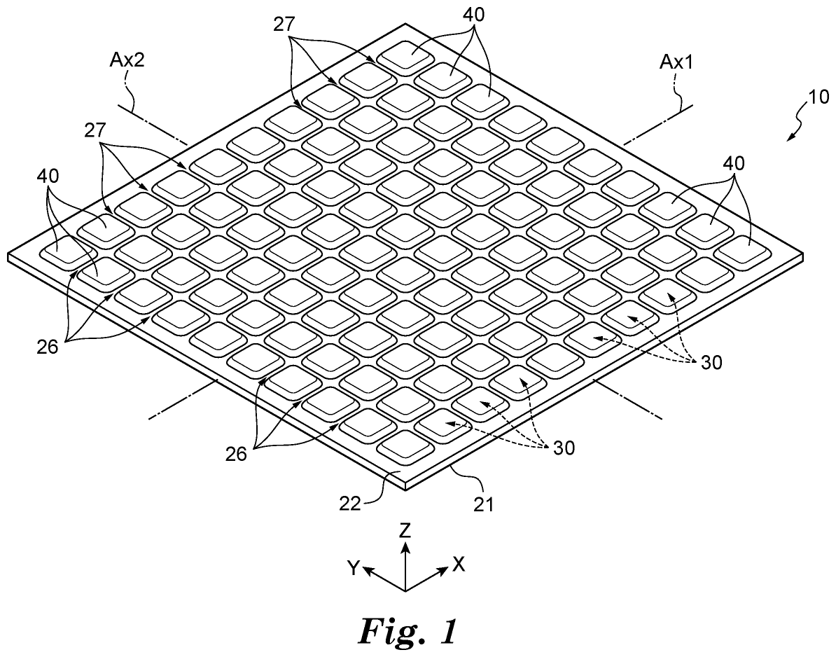

Detailed description of embodiments of the present invention will be given below with reference to the accompanied drawings. Note that, in the following description, the same elements or equivalents are denoted by the same reference sings, and redundant description of these elements will be omitted. In the embodiments, each of an axis X, an axis Y, and an axis Z is set in FIGS. 1 to 7B, but each of these axes is set for convenience of description.

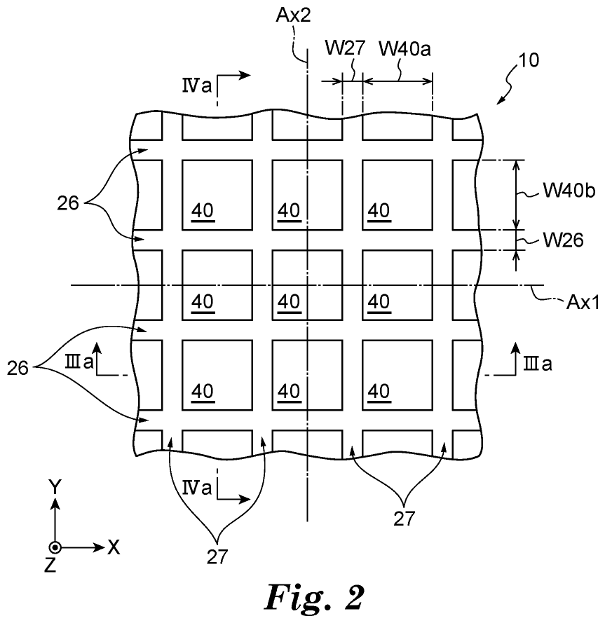

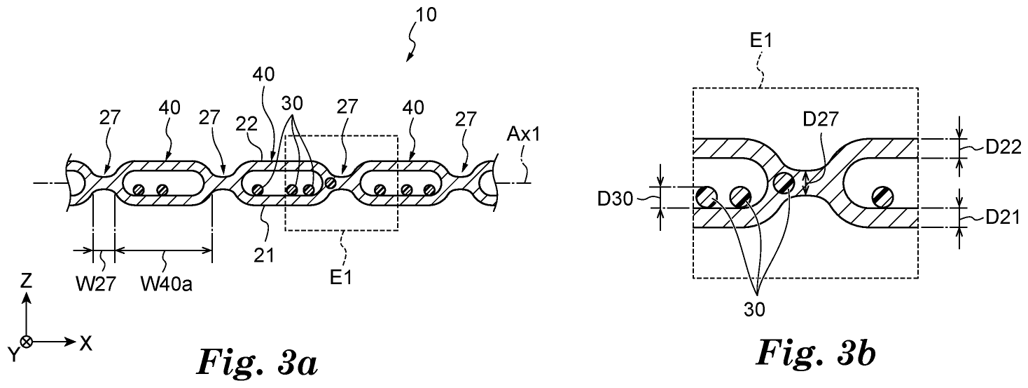

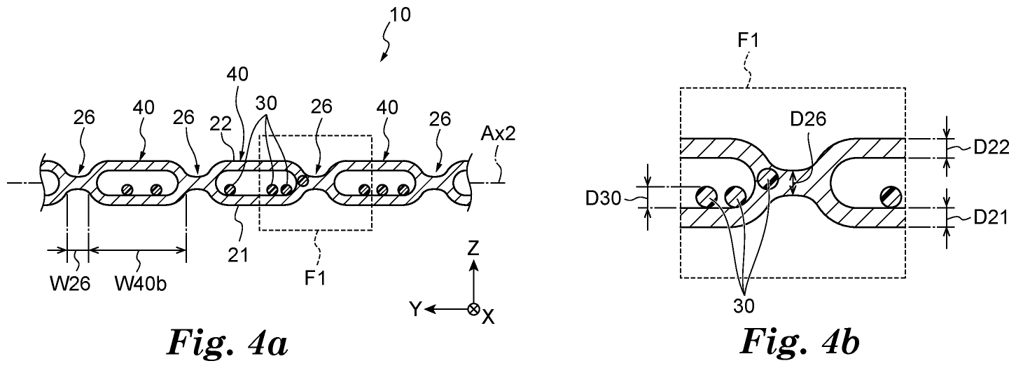

FIG. 1 is a perspective view schematically illustrating an absorbent body according to the embodiment. FIG. 2 is a plan view schematically illustrating the absorbent body according to the embodiment. FIG. 3A is a cross-sectional view taken along line of FIG. 2, and FIG. 3B is an enlarged cross-sectional view of a region E1 of FIG. 3A. FIG. 4A is a cross-sectional view taken along line IVa-IVa of FIG. 2, and FIG. 4B is an enlarged cross-sectional view of a region F1 of FIG. 4A. FIGS. 1 to 4B illustrate an absorbent body 10 obtained before water absorption.

The absorbent body 10 includes a first elastic member 21, a second elastic member 22, and an absorbing member 30. The absorbing member 30 is laminated between the first elastic member 21 and the second elastic member 22. The first elastic member 21 forms, together with the second elastic member 22, a plurality of housing parts 40. The plurality of housing parts 40 house the absorbing member 30.

A type of the absorbing member 30 is not particularly limited as long as the material is excellent in water absorbency. The absorbing member 30 includes, for example, a water absorbing polymer. In addition, the absorbing member 30 may include a water absorbing polymer and fiber holding the water absorbing polymer such as polyolefin non-woven fabric and cotton-like pulp.

The water absorbing polymer includes, for example, a material such as sodium polyacrylate, an acrylic acid-vinyl alcohol copolymer, a sodium polyacrylate cross-linked product, a starch-acrylic acid graft polymer, or an isobutylene-maleic anhydride. The water absorbing polymer can be a Super Absorbent Polymer (SAP) absorbing 10 or more times its own weight of water. The fiber holding the water absorbing polymer include, for example, carboxymethylcellulose.

At least one of the first elastic member 21 and the second elastic member 22 can include non-woven fabric. In the present embodiment, both the first elastic member 21 and the second elastic member 22 can include non-woven fabric. In addition, one of the first elastic member 21 and the second elastic member 22 can include non-woven fabric, and the other of the first elastic member 21 and the second elastic member 22 can include any of an elastic film and woven fabric. Both the first elastic member 21 and the second elastic member 22 can include any of an elastic film and woven fabric.

The non-woven fabric includes, for example, polypropylene fiber, mixed fiber of polyester fiber and polyolefin fiber, or composite fiber of polyethylene terephthalate and polyethylene. The non-woven fabric is produced, for example, by a method such as a spunbond method, a spunlace method, or a thermal bond method. The non-woven fabric can include a material processable into fiber, and in addition, the non-woven fabric has various kinds of flexibility, for example, by adjusting a combination of contained fiber and/or by adjusting a fiber length, a thickness of fiber, and the like.

The elastic film includes, for example, an elastomer. A type of the elastomer is not particularly limited. The elastomer, for example, includes a resin such as a styrene-isoprene-styrene block copolymer (SIS), a styrene-butadiene-styrene block copolymer (SBS), a styrene-ethylene/butylene-styrene block copolymer (SEBS), polyurethane, an ethylene copolymer, or a propylene oxide (PO). These resins can be thermoplastic resins. The ethylene copolymer includes, for example, ethylene vinyl acetate, an ethylene-propylene copolymer, or an ethylene-propylene-diene terpolymer. The elastic film may further include, for example, polyolefin in addition to the elastomer.

The woven fabric includes, for example, synthetic fiber, natural fiber, or a combination of synthetic fiber and natural fiber. The synthetic fiber present in the woven fabric include, for example, nylon fiber, vinylon fiber, polypropylene fiber, polyester fiber, or ultrahigh molecular weight polyethylene fiber. The natural fiber present in the woven fabric include, for example, cotton or hemp.

At least one of the first elastic member 21 and the second elastic member 22 can have liquid permeability. Namely, both the first elastic member 21 and the second elastic member 22 may have liquid permeability. In addition, any one of the first elastic member 21 and the second elastic member 22 may have liquid permeability, and the other of the first elastic member 21 and the second elastic member 22 may have no liquid permeability. Due to the liquid permeability of these elastic members, liquid such as urine can infiltrate inside of the absorbent body 10 via at least one of the first elastic member and the second elastic member 22, and the absorbing member 30 housed inside of the absorbent body 10 can absorb the liquid infiltrating.

The non-woven fabric has elasticity, and the elastic non-woven fabric has various kinds of flexibility by adjusting a fiber length and a thickness of fiber. In addition, since the elastic non-woven fabric can also have liquid permeability, the absorbent body 10 can suitably include the elastic non-woven fabric. The absorbent body 10 including the elastic non-woven fabric can follow various postures of a wearer of the absorbent body 10 and in addition, can sufficiently absorb liquid such as urine from the wearer.

The absorbent body 10 includes a first delimiting part 26 and a second delimiting part 27, and the plurality of housing parts 40 are delimited from each other by the first delimiting part 26 and the second delimiting part 27. The first delimiting part 26 is along a first direction Ax1, and the second delimiting part 27 is along a second direction Ax2 intersecting the first direction Ax1. As illustrated in FIGS. 1 and 2, the first direction Ax1 is, for example, along the X axis, and the second direction Ax2 is, for example, along the Y axis. The plurality of housing parts 40 are provided, for example, to be two-dimensionally arrayed in a lattice.

The first delimiting part 26 and the second delimiting part 27 define the plurality of housing parts 40 formed by joining the first elastic member 21 and the second elastic member 22, and define outer edges of the plurality of housing parts 40. Namely, the plurality of housing parts 40 are surrounded by the first delimiting part 26 and the second delimiting part 27 and the outer edges of the plurality of housing parts 40 are defined. Note that, the first delimiting part 26 and the second delimiting part 27 may have a configuration in which the first delimiting part 26 and the second delimiting part 27 extend continuously (namely, in an uninterrupted state), or may extend intermittently in a perforation-like line. The state in which the first delimiting part 26 and the second delimiting part 27 extend intermittently is a state in which a joined portion where the first elastic member 21 and the second elastic member 22 are joined together and a non-joined portion where the first elastic member 21 and the second elastic member 22 are not joined together are alternately present. According to such a configuration, since the first elastic member 21 and the second elastic member 22 are made elastic at the non-joined portion where the first elastic member 21 and the second elastic member 22 are not fixed, elasticity of the absorbent body 10 as a whole improves. However, in a case where the first delimiting part 26 and the second delimiting part 27 extend intermittently, a size of the non-joined portion is set to a size in which the absorbing member 30 in the housing parts 40 dose not escape from the non-joined portion, or, is set to a size within an allowable extent even when the absorbing member 30 escapes.

The first delimiting part 26 and the second delimiting part 27 define array of the plurality of housing parts 40. Namely, in the first direction Ax1, the housing parts 40 are arrayed to be sandwiched by a pair of second delimiting parts 27. In addition, in the second direction Ax2, the housing parts 40 are arrayed to be sandwiched by a pair of first delimiting parts 26. In the first direction Ax1, the housing parts 40 and the second delimiting parts 27 are alternately arrayed, and in the second direction Ax2, the housing parts 40 and the first delimiting parts 26 are alternately arrayed. The absorbent body 10 has alternating array of the housing parts 40 and the first delimiting parts 26 or the second delimiting parts 27 both in the first direction Ax1 and the second direction Ax2.

The housing parts 40 are each a space surrounded by the first elastic member 21 and the second elastic member 22 and housing the absorbing member 30. The first elastic member 21 and the second elastic member 22 are joined to each other at the first delimiting part 26 and the second delimiting part 27, and the housing parts 40 are each surrounded by the first elastic member 21 and the second elastic member 22 except for a portion corresponding to the first delimiting part 26 and the second delimiting part 27. Since the housing parts 40 can each form a space closed by the first elastic member 21 and the second elastic member 22, the absorbing member 30 can be housed not to fall off from the housing parts 40. In each of the housing parts 40, the absorbing member 30 is not fixed to the first elastic member 21 and the second elastic member 22 by an adhesive, and is not fixed to each other between absorbing members 30. Namely, the absorbing member 30 can freely move within the range of an internal space of each of the housing parts 40.

In the present embodiment, the housing parts 40 each have, for example, a square two-dimensional shape. In addition, the housing part 40 has, in addition to a square, for example, a quadrilateral two-dimensional shape such as a rectangle, a rhombus, and a trapezoid. The housing parts 40 may each have, in addition to a quadrilateral, a polygonal two-dimensional shape such as a triangle and a pentagon. In addition, the housing parts 40 are not limited to the two-dimensional shape surrounded by a straight line like a polygon, and may each have a two-dimensional shape surrounded by a curve such as a circular arc and a wave line.

As illustrated in FIGS. 3A, 3B, 4A and 4B, the absorbing member 30 is laminated between the first elastic member 21 and the second elastic member 22. In addition, for example, the absorbing member 30 is laminated between the first elastic member 21 and the second elastic member 22, for example, to an extent that the absorbing member 30 can be regarded as uniformly laminated between the first elastic member 21 and the second elastic member 22, and to include a region in which the first delimiting part 26 and the second delimiting part 27 are formed. Here, being uniformly laminated means that the absorbing member 30 is evenly laminated between the first elastic member 21 and the second elastic member 22 without distinguishing the region in which the first delimiting part 26 and the second delimiting part 27 are formed from regions other than this region. In addition, laminating means disposing the absorbing member 30 on the first elastic member 21 and/or the second elastic member 22.

At least one of the first delimiting part 26 and the second delimiting part 27 can include the absorbing member 30 in the region of at least a portion of the first delimiting part 26 and the second delimiting part 27. Namely, both of the first delimiting part 26 and the second delimiting part 27 can include the absorbing member 30 in almost all region of the first delimiting part 26 and the second delimiting part 27. In addition, any one of the first delimiting part 26 and the second delimiting part 27 can be set to include the absorbing member 30 in almost all the region of any one of the first delimiting part 26 and the second delimiting part 27, and the other one of the first delimiting part 26 and the second delimiting part 27 can be set not to include the absorbing member 30 in almost all the region of the other one of the first delimiting part 26 and the second delimiting part 27.

The first delimiting part 26 and the second delimiting part 27 can both include the absorbing member 30 in the region of a portion of the first delimiting part 26 and the second delimiting part 27. For example, out of a plurality of the first delimiting parts 26 and a plurality of the second delimiting parts 27, only a portion of the first delimiting parts 26 and a portion of the second delimiting parts 27 may include the absorbing member 30, and other portions of the first delimiting parts 26 and other portions of the second delimiting parts 27 may not include the absorbing member 30. In addition, with regard to each one of the first delimiting parts 26 and each one of the second delimiting parts 27, the absorbing member 30 may not be present over the entire length in a longitudinal direction of each of the first delimiting part 26 and the second delimiting part 27, or the absorbing member 30 may be present in a region of a portion in the longitudinal direction of the first delimiting part 26 and the second delimiting part 27, and may not be present in other regions.

In addition to being laminated to an extent that the absorbing member 30 can be regarded as uniformly laminated between the first elastic member 21 and the second elastic member 22, the absorbing member 30 may be laminated to have density varied of the absorbing member 30. For example, an amount of the absorbing member 30 to be laminated may be made smaller in the region in which the first delimiting part 26 and the second delimiting part 27 are formed as compared to the region in which the first delimiting part 26 and the second delimiting part 27 are not formed. For example, in addition to being laminated on the first elastic member 21, the absorbing member 30 may be laminated to be held in a material such as non-woven fabric present in the first elastic member 21.

In the present embodiment, the housing parts 40 each have a first width W40a along the first direction Ax1 and a second width W40b along the second direction Ax2. In addition, the first delimiting part 26 has a third width W26 along the second direction Ax2, and the second delimiting part 27 has a fourth width W27 along the first direction Ax1.

The first width W40a of the housing part 40 is, for example, from 3 to 50 mm, and the second width W40b of the housing part 40 is, for example, from 3 to 50 mm. The third width W26 of the first delimiting part 26 is, for example, from 0.1 to 10 mm, and the fourth width W27 of the second delimiting part 27 is, for example, from 0.1 to 10 mm.

In addition, in the present embodiment, a first thickness D21 of the first elastic member 21 is, for example, from 0.01 to 5 mm, and a second thickness D22 of the second elastic member 22 is, for example, from 0.01 to 5 mm. The first elastic member 21 and the first elastic member 21 both include, for example, non-woven fabric. A third thickness D26 of the first delimiting part 26 is, for example, from 0.01 to 5 mm, and a fourth thickness D27 of the second delimiting part 27 is, for example, from 0.01 to 5 mm. The absorbing member 30 has a fifth thickness D30 in a thickness direction of the first elastic member 21. The fifth thickness D30 is, for example, from 0.01 to 1 mm. The absorbing member 30 includes, for example, a water absorbing polymer.

At least one of the first delimiting part 26 and the second delimiting part 27 can include a welded portion 28. Since the first elastic member 21 and the second elastic member 22 are joined to each other by welding, the joining can be firm. Due to this firm joining, the absorbent body 10 in one housing part 40 is difficult to move to another housing part 40 before and after water absorption of the absorbent body 10, and in addition, is difficult to fall off from the one housing part 40. The “welding” means that the first elastic member 21 and the second elastic member 22 are joined to each other by melting the first elastic member 21 and the second elastic member 22.

As illustrated in FIGS. 3A, 3B, 4A, and 4B, the absorbing member 30 is laminated between the first elastic member 21 and the second elastic member 22 to make volume of the housing part 40 larger than volume of the absorbing member 30. Here, the volume of the housing part 40 means volume of one space surrounded by the first elastic member 21 and the second elastic member 22. The volume of the absorbing member 30 means a sum of the volume of absorbing members 30. Therefore, when there is one absorbing member 30, the volume of the absorbing member 30 means volume of the one absorbing member 30, and when there are a plurality of the absorbing members 30, the volume of the absorbing member 30 means a sum of the volume of the plurality of absorbing members 30.

In the present embodiment, the volume of the housing part 40 before water absorption is, for example, from 0.09 to 25000 mm³, and volume of the water absorbing polymer which is one of the absorbing members 30 before water absorption is, for example, from 1×10′ to 24000 mm³. The first elastic member 21 and the second elastic member 22 both include, for example, non-woven fabric, and the absorbing member 30 includes, for example, the water absorbing polymer.

In the present embodiment, when the first elastic member 21 and the first elastic member 21 include non-woven fabric, a thickness of each of the first elastic member 21 and the first elastic member 21 reduces as compared to, for example, when the first elastic member 21 and the first elastic member 21 include a material such as pulp, and thus, wearing feeling of a wearer of the absorbent body 10 improves. In addition, when the thickness of the elastic member is small, the volume of the absorbent body 10 decreases, and thus, it becomes easier to carry the absorbent body 10.

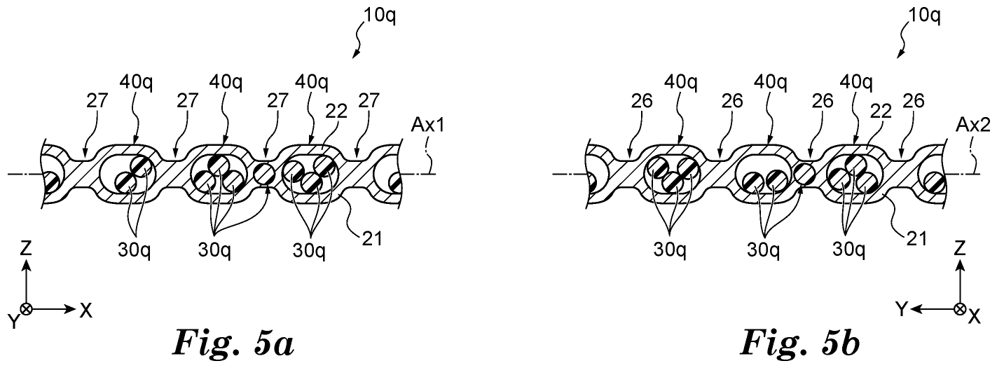

FIG. 5A is a cross-sectional view corresponding to FIG. 3A according to the embodiment, and FIG. 5B is a cross-sectional view corresponding to FIG. 4A according to the embodiment. FIGS. 3A and 4A illustrate the absorbent body 10 obtained before water absorption, and FIGS. 5A and 5B illustrate an absorbent body 10q obtained after water absorption.

The absorbing member 30 has volume before water absorption smaller than the volume of the housing part 40, and in addition, even though volume of an absorbing member 30q obtained after water absorption is increased, the absorbing member 30q obtained after water absorption still can be held in a housing part 40q obtained after water absorption. In the present embodiment, volume of the housing part 40q obtained after water absorption is, for example, from 0.9 to 1.5×10⁶ mm³, and the volume of the absorbing member 30q obtained after water absorption is, for example, from 1×10′ to 1.4×10⁶ mm³. The first elastic member 21 and the first elastic member 21 both include, for example, non-woven fabric, and the absorbing member 30q includes, for example, a water absorbing polymer. The absorbing member 30 housed in the first delimiting part 26 and the second delimiting part 27 also absorbs urine and the like of a wearer and volume of the absorbing member 30 increases.

At least one of the first elastic member 21 and the second elastic member 22 houses the absorbing member 30 obtained before and after water absorption to prevent the absorbing member 30 from falling off from the housing part 40, and in addition, at least one of the first elastic member 21 and the second elastic member 22 can have elasticity to enable the absorbing member 30 obtained before and after water absorption to follow various postures of a wearer of the absorbing member 30 obtained before and after water absorption. In the present embodiment, at least one of the first elastic member 21 and the second elastic member 22 has, for example, elasticity of a load from 0.1 to 10 N/25 mm when 50% elongation is performed at a speed of 300 mm/minute. Since both the first elastic member 21 and the second elastic member 22 can have elasticity, the absorbing member 30q housed in the housing parts 40, for example, a water absorbing polymer becomes easy to undergo swelling due to water absorption.

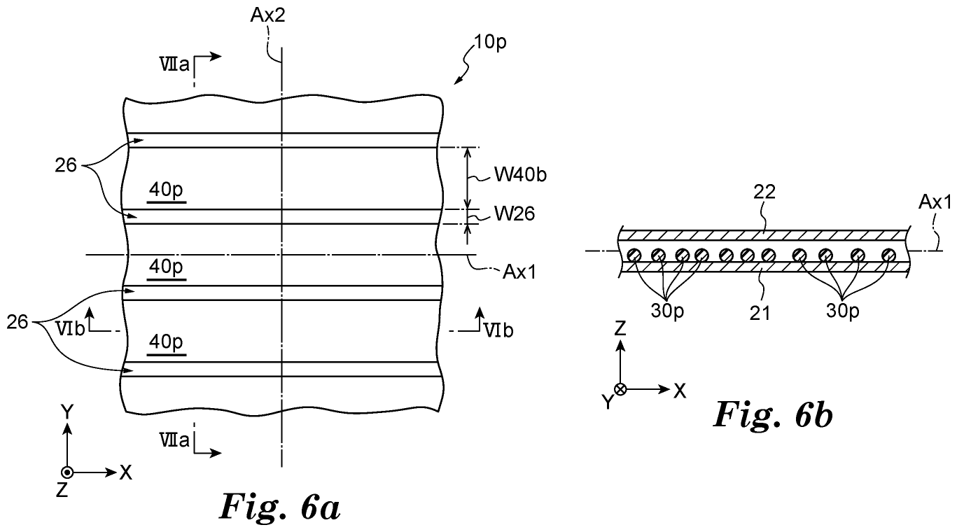

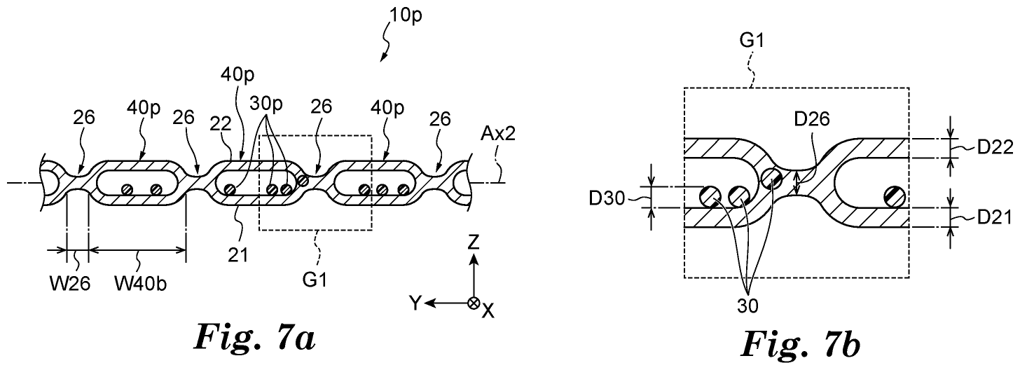

Here, an absorbent body according to a comparative example will be described for comparison. FIG. 6A is a plan view schematically illustrating an absorbent body according to a comparative example. FIG. 6B is a cross-sectional view taken along line VIb-VIb of FIG. 6A. FIG. 7A is a cross-sectional view taken along line VIIa-VIIa of FIG. 6A, and FIG. 7B is an enlarged cross-sectional view of a region G1 of FIG. 7A.

An absorbent body 10p according to the comparative example includes a first delimiting part 26p along a first direction Ax1, and a plurality of housing parts 40p are sandwiched by a pair of first delimiting parts 26b, and are delimited from each other by the first delimiting parts 26b. The plurality of housing parts 40p extend in the first direction Ax1. The absorbent body 10p has alternating array of the housing parts 40p and the first delimiting parts 26b in a second direction Ax2.

In the absorbent body 10p according to the comparative example, the plurality of housing parts 40 are delimited from each other only by, for example, the first delimiting parts 26 along the first direction Ax1. Therefore, each of the housing parts 40p between the pair of first delimiting parts 26 is very long in the first direction Ax1. When the absorbing member 30 is disposed in such a housing part 40p without using an adhesive, the absorbing member 30 widely moves in the first direction Ax1, and thus the absorbing member 30 cannot be held and kept at a desired position. Therefore, in the absorbent body 10p according to the comparative example, it is necessary, for example, to apply an adhesive to the first elastic member 21 and fix the absorbing member 30 by the adhesive. In such an absorbent body 10p, when the absorbing member 30 is fixed by using an adhesive, the elastic member becomes hard, and thus elasticity and flexibility are impaired, and in addition, water absorption performance of the absorbing member 30 also reduces due to hardening the absorbing member 30 by the adhesive.

In contrast, the absorbent body 10 according to the present embodiment includes the plurality of housing parts 40, and the plurality of housing parts 40 are delimited from each other by the first delimiting part 26 along the first direction Ax1 and by the second delimiting part 27 along the second direction Ax2. In this manner, since the housing parts 40 are delimited in directions intersecting each other, the absorbing member 30 can be held at a predetermined position without fixing the absorbing member 30 by an adhesive. In this manner, since no adhesive for fixing the absorbing members 30 can be necessary, the first elastic member 21 and the second elastic member 22 can have elasticity in a plurality of directions in a state in which loss of elasticity is suppressed, and flexibility of the first elastic member 21 and the second elastic member 22 is maintained.

In addition, the absorbent body 10 according to the present embodiment includes the first elastic member 21, the second elastic member 22, and the absorbing member 30 laminated between the first elastic member 21 and the second elastic member 22. In this manner, since both of the members sandwiching the absorbing member 30 are elastic members, the absorbent body can have elasticity in a plurality of directions. In addition, since the absorbing member 30 is not fixed by the adhesive, reduction in water absorption performance of the absorbing member 30 due to hardening the absorbing member 30 by the adhesive can also be prevented.

Note that, in the first direction Ax1, the housing parts 40 are each sandwiched by the pair of second delimiting parts 27, and in addition, in the second direction Ax2, the housing parts 40 are each sandwiched by the pair of first delimiting parts 26. In the first direction Ax1, the housing parts 40 and the second delimiting parts 27 are alternately arrayed, and in the second direction Ax2, the housing parts 40 and the first delimiting parts 26 are alternately arrayed. Since the absorbent body 10 has alternating array in both of the first direction Ax1 and the second direction Ax2 of the housing parts 40 and the first delimiting parts 26 or the second delimiting parts 27, elasticity of the absorbent body 10 in the first direction Ax1 becomes similar to elasticity of the absorbent body 10 in the second direction Ax2. Since the absorbent body 10 according to an embodiment has elasticity similar in a plurality of directions of the first direction Ax1 and the second direction Ax2 intersecting the first direction Ax1, the absorbent body 10 can easily follow various postures of a wearer, and can reduce a gap with a skin of the wearer.

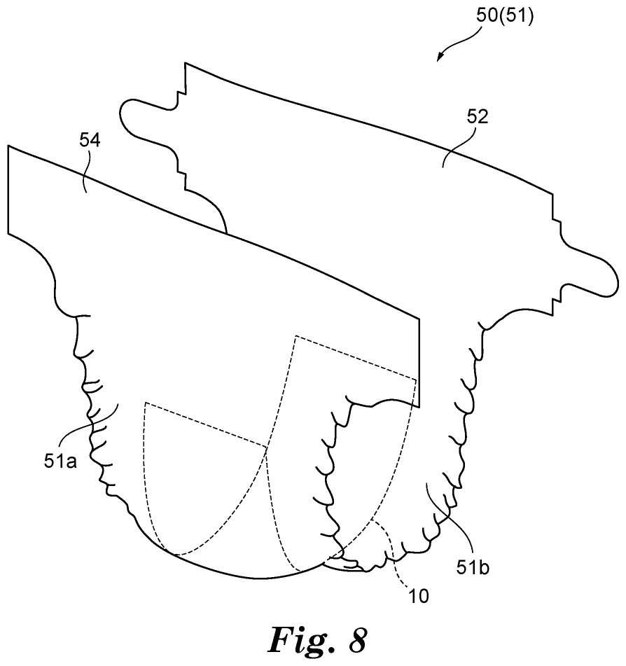

FIG. 8 is a view illustrating an absorbent article including an absorbent body. Specifically, an absorbent article 50 includes a hygiene article such as a diaper, a napkin, or a urine absorbing pad, and FIG. 8 schematically illustrates a diaper 51 as an example of the absorbent article 50. The diaper 51 includes an absorbent body 10 obtained before water absorption.

The diaper 51 includes the absorbent body 10, a top sheet 52, and a back sheet 54. The absorbent body 10 is housed between the top sheet 52 and the back sheet 54. The top sheet 52 has liquid permeability and is laminated inside of the back sheet 54. Liquid such as urine from a wearer of the diaper 51 reaches the absorbent body 10 via the top sheet 52, and is absorbed by the absorbent body 10. The back sheet 54 includes a laminate of a liquid impermeable film and a non-woven fabric layer. The liquid impermeable film is provided on the absorbent body 10 side, and the non-woven fabric layer is provided on an outer surface 51a side of the diaper 51. When the diaper 51 is worn, the top sheet 52 is disposed on an inner surface 51b side of the diaper 51 adjacent to a skin of the wearer or disposed on the side close to the skin of the wearer, and the non-woven fabric layer of the back sheet 54 is disposed on the outer surface 51a side of the diaper 51 adjacent to cloth or the like of the wearer, or disposed on the side close to the cloth of the wearer.

In the diaper 51, the non-woven fabric layer used for the back sheet 54 can be manufactured by a manufacturing method such as a spunbond method, for example. Mass of the non-woven fabric layer is, for example, from 15 to 40 g/m², preferably from 25 to 35 g/m². Fiber density is, for example, from 0.06 to 0.1 g/cm³, preferably from 0.07 to 0.09 g/cm³. The non-woven fabric layer includes, for example, non-woven fabric such as polyolefin and PET. The absorbent article 50 such as the diaper 51 according to the present embodiment can follow various postures of the wearer.

As illustrated in FIGS. 3A to 5B, in the absorbent body 10 obtained before water absorption and the absorbent body 10q obtained after water absorption, each of the first elastic member 21 and the second elastic member 22 has a concave/convex shape along a thickness direction (Z direction). Namely, the absorbent body 10 obtained before water absorption and the absorbent body 10q obtained after water absorption form convex portions in the thickness direction, and the first delimiting parts 26 and the second delimiting parts 27 form concave portions in the thickness direction. Due to this concave/convex shape, in the absorbent body 10 obtained before water absorption and the absorbent body 10q obtained after water absorption, a surface opposite to a surface being in contact with the wearer can have a passage of air, and thus heat and water vapor from the absorbent body easily diffuse in a horizontal direction (for example, the Y direction) via the passage. In addition, due to the concave/convex shape, the absorbent body 10 obtained before water absorption and the absorbent body 10q obtained after water absorption can reduce adhesion area to the back sheet 54 of the diaper 51, and thus the diaper 51 has high bendability and flexibility.

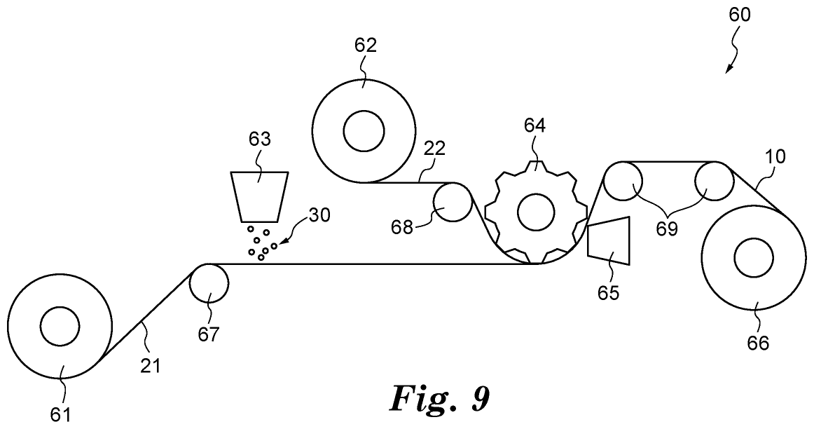

FIG. 9 is a view schematically illustrating an absorbent body manufacturing apparatus. An absorbent body manufacturing apparatus 60 includes a first extrusion roll 61, a second extrusion roll 62, an absorbing member laminating unit 63, a laminating drum 64, a joining device 65, and a winding roll 66.

The first extrusion roll 61 is a device for holding the first elastic member 21 and for extruding the first elastic member 21 toward the laminating drum 64, and the second extrusion roll 62 is a device for holding the second elastic member 22 and for extruding the second elastic member 22 toward the laminating drum 64. The absorbing member laminating unit 63 is a device for laminating the absorbing member 30 on the first elastic member 21. The laminating drum 64 and the joining device 65 are devices for joining in cooperation the first elastic member 21 and the second elastic member 22 to form the plurality of housing parts 40. The laminating drum 64 is a device that also extrudes toward the winding roll 66 the first elastic member 21 and the second elastic member 22 joined. The winding roll 66 is a device for winding up the absorbent body 10 in which the plurality of housing parts 40 are formed.

The absorbent body manufacturing apparatus 60 further includes a first conveying roll 67, a second conveying roll 68, and a third conveying roll 69. The first conveying roll 67 is a device for conveying the first elastic member 21 from the first extrusion roll 61 toward the laminating drum 64, and the second conveying roll 68 is a device for conveying the second elastic member 22 from the first extrusion roll 61 toward the laminating drum 64. The third conveying roll 69 is a device for conveying from the laminating drum 64 toward the winding roll 66 the absorbent body 10 on which the plurality of housing parts 40 are formed.

The absorbing member laminating unit 63 is disposed, for example, between the first conveying roll 67 and the laminating drum 64. The first elastic member 21 can be conveyed in the order of the first extrusion roll 61, the first conveying roll 67, the absorbing member laminating unit 63, the laminating drum 64, the joining device 65, the third conveying roll 69, and the winding roll 66. The second elastic member 22 can be conveyed in the order of the second extrusion roll 62, the second conveying roll 68, the absorbing member laminating unit 63, the laminating drum 64, the joining device 65, the third conveying roll 69, and the winding roll 66.

Examples of a method of joining the first elastic member 21 and the second elastic member 22 include a thermal welding method and an ultrasonic welding method.

In the thermal welding method, when the first elastic member 21 and the second elastic member 22 pass between the joining device 65 and the laminating drum 64, the first elastic member 21 and the second elastic member 22 receive heat energy from the joining device 65 and melt/weld each other. The joining device 65 in the thermal welding method includes a heating unit 65a. After the first elastic member 21 and the second elastic member 22 are melted, the first elastic member 21 and the second elastic member 22 are pressed against the joining device 65 and the laminating drum 64 to receive pressure until the first elastic member 21 and the second elastic member 22 are firmly joined to each other by welding.

The first delimiting part 26 and the second delimiting part 27 are produced by joining the first elastic member 21 and the second elastic member 22, and the plurality of housing parts 40 are formed as a result of producing the delimiting parts. The plurality of housing parts 40 are delimited from each other by the first delimiting part 26 and the second delimiting part 27.

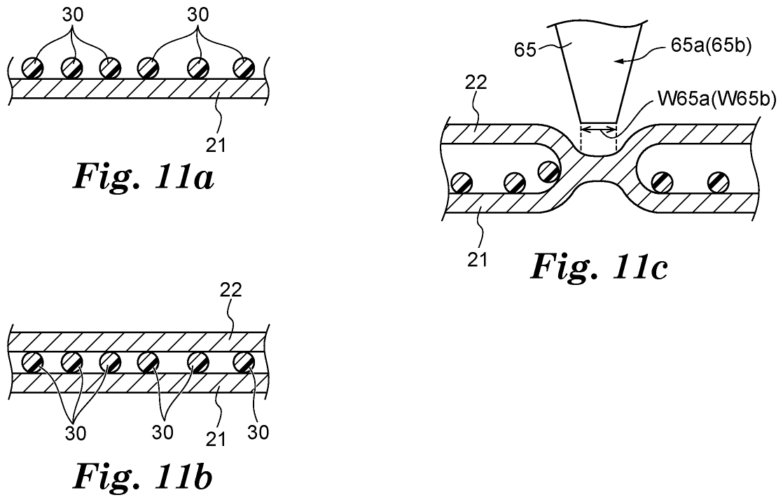

A tip of the heating unit 65a has, for example, a shape that can form the plurality of housing parts 40 where the first dividing unit 26 and the second dividing unit 27 are two-dimensionally arrayed in a lattice. In the thermal welding method, for example, the first elastic member 21 and the second elastic member 22 are melted and welded by bringing the heating unit 65a (see FIG. 11C) such as a heated metal plate into contact with a joint part between the first elastic member 21 and the second elastic member 22.

In the ultrasonic welding method, the first elastic member 21 and the second elastic member 22 are melted and welded by passing the first elastic member 21 and the second elastic member 22 between the joining device 65 and the laminating drum 64. The joining device 65 includes a horn 65b (see FIG. 11C).

In the ultrasonic welding method, a converter converts high frequency electrical energy from an ultrasonic oscillator into mechanical vibration energy, and the converted mechanical vibration energy is sent through the horn 65b to the joint part between the first elastic member 21 and the second elastic member 22. As a result, when the first elastic member 21 and the second elastic member 22 pass between the horn 65b and the laminating drum 64, the first elastic member 21 and the second elastic member 22 receive the mechanical vibration energy from the horn 65b to generate strong frictional heat, and melt and weld each other.

A tip of the horn 65b has, for example, a shape enabling forming the plurality of housing parts 40 where the first delimiting part 26 and the second delimiting part 27 are two-dimensionally arrayed in a lattice. A width of the tip of the horn 65b can be sized to match a width of the first delimiting part 26 and a width of the second delimiting part 27.

Both the thermal welding method and the ultrasonic welding method can firmly join the first elastic member 21 and the second elastic member 22 together. Due to this firm joining, the absorbent body 10 in one housing part 40 is difficult to move to another housing part 40 before and after water absorption of the absorbent body 10, and in addition, is difficult to fall off from the one housing part 40.

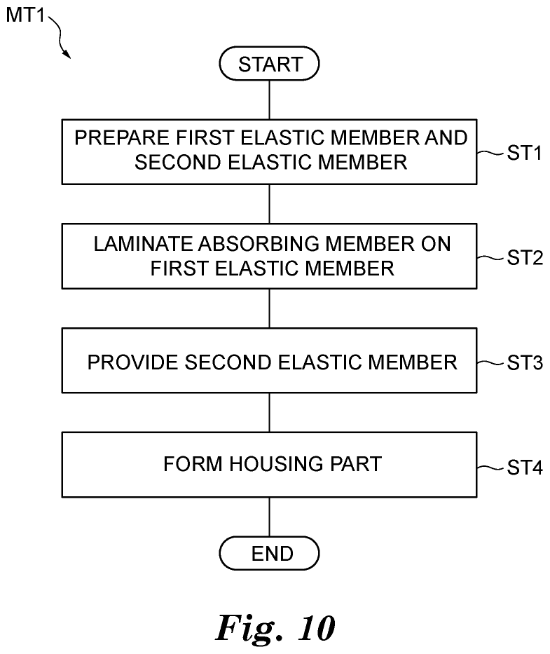

FIG. 10 is a flow chart illustrating a method of manufacturing an absorbent body according to an embodiment. FIGS. 11A, 11B, and 11C are views illustrating products at steps in the method of manufacturing an absorbent body, respectively. Description of a manufacturing method MT1 of an absorbent body will be made with reference to the absorbent body manufacturing apparatus 60 illustrated in FIG. 9.

In the manufacturing method MT1 of the absorbent body 10, first, the first elastic member 21 and the second elastic member 22 are prepared (step ST1). Specifically, at step ST1, the first elastic member 21 and the second elastic member 22 are wound around the first extrusion roll 61 and the second extrusion roll 62, respectively. Then, the first elastic member 21 is conveyed by the first extrusion roll 61 and the first conveying roll 67 toward the laminating drum 64. The second elastic member 22 is conveyed by the second extrusion roll 62 and the second conveying roll 68 toward the laminating drum 64.

In the manufacturing method MT1, the absorbing member 30 is laminated on the first elastic member 21 before the first elastic member 21 is conveyed to the laminating drum 64 (step ST2). As illustrated in FIG. 11A, lamination of the absorbing member 30 is performed, for example, by spraying the absorbing member 30 from the absorbing member laminating unit 63. The absorbing member 30 is sprayed, for example, to an extent that the absorbing member 30 can be regarded as uniformly sprayed on the first elastic member 21, and to include a region in which the first delimiting part 26 and the second delimiting part 27 are formed at a subsequent step. Being uniformly sprayed means that the absorbing member 30 is evenly sprayed on the first elastic member 21 without distinguishing the region in which the first delimiting part 26 and the second delimiting part 27 are formed from other regions.

After the absorbing member 30 is laminated on the first elastic member 21, the second elastic member 22 is provided on the first elastic member 21 and on the absorbing member 30 laminated on the first elastic member 21 (step ST3). Namely, both the first elastic member 21 and the second elastic member 22 are conveyed to the laminating drum 64, and subsequently, the second elastic member 22 is provided on the first elastic member 21 by the laminating drum 64. At step ST3, as illustrated in FIG. 11B, since the absorbing member 30 is laminated on the first elastic member 21 by the absorbing member laminating unit 63, the second elastic member 22 is also provided on this absorbing member 30.

In the manufacturing method MT1, after the second elastic member 22 is provided, the plurality of housing parts 40 are formed (step ST4). At step ST4, when a laminate of the first elastic member 21, the absorbing member 30 laminated on the first elastic member 21, and the second elastic member 22 passes between the joining device 65 and the laminating drum 64, the first elastic member 21 and the second elastic member 22 are joined to each other and the first delimiting part 26 and the second delimiting part 27 are produced. As a result of the production of these delimiting parts, the plurality of housing parts 40 are formed, and the plurality of housing parts 40 are delimited from each other by the first delimiting part 26 and the second delimiting part 27. The plurality of housing parts 40 house the absorbing member 30.

At step ST4, the delimiting parts can be produced by a thermal welding method or an ultrasonic welding method. As illustrated in FIG. 11C, at the time of the production of the delimiting parts, the tip of the heating unit 65a or the tip of the horn 65b approaches or comes into contact with the joint part between the first elastic member 21 and the second elastic member 22, and heat or an ultrasonic wave is supplied to the first elastic member 21. The first elastic member 21 and the second elastic member 22 melt together and as a result, weld each other by the supply of heat or an ultrasonic wave.

In the production of the delimiting parts by the thermal welding method, a width of the heating unit 65a such as a metal plate can be sized to match a width of the first delimiting part 26 and a width of the second delimiting part 27. Specifically, a width W65a of the heating unit 65a is, for example, from 0.01 to 5 mm. In the production of the delimiting parts by the ultrasonic welding method, a width W65b of the tip of the horn 65b is, for example, from 0.01 to 5 mm.

According to the manufacturing method MT1 of an absorbent body according to the present embodiment, since the absorbing member 30 is sandwiched between the first elastic member 21 and the second elastic member 22 that are elastic members, the absorbent body 10 produced can have elasticity in a plurality of directions. The absorbent body 10 includes the plurality of housing parts 40, and the plurality of housing part 40 are delimited from each other by the first delimiting part 26 along the first direction Ax1 and the second delimiting part 27 along the second direction Ax2 intersecting the first direction Ax1. In this manner, since the housing parts 40 are delimited in directions intersecting each other, the absorbing member 30 can be held at a predetermined position without fixing the absorbing member 30 by an adhesive. In this manner, since no adhesive for fixing the absorbing member 30 may be used, the first elastic members 21 and the second elastic members 22 can have elasticity in a plurality of directions in a state in which loss of elasticity is prevented.

When the first elastic member 21 and the second elastic member 22 are joined to each other by welding, flexibility of the absorbent body 10 improves as compared to, for example, when the first elastic member 21 and the second elastic member 22 are joined to each other by an adhesive. The joining by welding can prevent the adhesive from adhering to surfaces of the first elastic member 21 and the second elastic member 22 other than the joint part, and thus liquid permeability of the first elastic member 21 and the second elastic member 22 improves. In addition, since adhesion of the adhesive to a surface of the absorbing member 30 can be avoided, water absorption area of the absorbing member 30 increases. The improvement of the liquid permeability and the increase of the water absorption area improve water absorption performance of the absorbent body 10.

In the manufacturing method MT1, the absorbing member 30 is uniformly sprayed on the first elastic member 21 to include the region in which the first delimiting part 26 and the second delimiting part 27 are formed, and thus the first delimiting part 26 and the second delimiting part 27 may include the absorbing member 30. At least one of the first delimiting part 26 and the second delimiting part 27 can include the absorbing member 30 in a region of at least a portion of the first delimiting part 26 and the second delimiting part 27.

In the manufacturing method MT1, at least one of the first delimiting part 26 and the second delimiting part 27 can include a welded portion. The welded portion can be formed by welding the first elastic member and the second elastic member by the thermal welding method or the ultrasonic welding method. Since the first delimiting part 26 and/or the second delimiting part 27 are formed by the thermal welding method or the ultrasonic welding method, the first elastic members 21 and the second elastic members 22 can have elasticity in a plurality of directions in a state in which loss of elasticity is prevented.

In the method of manufacturing the absorbent body 10, an adhesive can be used to form the first delimiting part 26 and the second delimiting part 27. In the method using the adhesive, for example, the adhesive is applied to the first elastic member 21 and the second elastic member 22 only in a region in which the first delimiting part 26 and the second delimiting part 27 are to be formed, and no adhesive is applied in a region in which the first delimiting part 26 and the second delimiting part 27 are not formed. According to this application of the adhesive, in the first elastic member 21 and the second elastic member 22 excluding the first delimiting part 26 and the second delimiting part 27, loss of elasticity is prevented, and elasticity in a plurality of directions can be provided.

When the first delimiting part 26 and the second delimiting part 27 are formed by welding, it is unnecessary to perform the spraying of the absorbing member 30 to avoid spraying an adhesive applied to the first elastic member 21 and/or the second elastic member 22, and thus the manufacturing steps can be simplified as compared to when the first delimiting part 26 and the second delimiting part 27 are formed by using the adhesive. In addition, since the absorbing member 30 housed in the first delimiting part 26 and the second delimiting part 27 absorbs urine and the like of a wearer, a water absorption amount of the absorbent body 10 can be increased.

### Embodiments

An absorbent body according to an embodiment of the present invention includes a first elastic member, a second elastic member, and an absorbing member laminated between the first elastic member and the second elastic member, and in the absorbent body, the first elastic member forms, together with the second elastic member, a plurality of housing parts, and the plurality of housing parts are delimited from each other by a first delimiting part along a first direction and a second delimiting part along a second direction intersecting the first direction, and house the absorbing member. An absorbent body having elasticity in a plurality of directions can be provided. An absorbent article including the absorbent body can be provided. In addition, a method of manufacturing the absorbent body having elasticity in a plurality of directions can be provided.

The absorbent body according to an embodiment includes the first elastic member, the second elastic member, and the absorbing member laminated between the first elastic member and the second elastic member. In this manner, since both the members sandwiching the absorbing member are elastic members, the absorbent body can have elasticity in a plurality of directions. Here, the absorbent body includes the plurality of housing parts, and the plurality of housing parts are delimited from each other by the first delimiting part along the first direction and the second delimiting part along the second direction intersecting the first direction. In this manner, since the housing parts are delimited in directions intersecting each other, the housing parts can hold the absorbing members at predetermined positions without fixing the absorbing members by an adhesive. In this manner, since no adhesive for fixing the absorbing members can be necessary, the first and second elastic members can have elasticity in a plurality of directions in a state in which loss of elasticity is suppressed.

In the absorbent body according to another embodiment, the absorbing member may include a water absorbing polymer.

In the absorbent body according to another embodiment, at least one of the first elastic member and the second elastic member may have liquid permeability.

In the absorbent body according to another embodiment, at least one of the first elastic member and the second elastic member may include non-woven fabric.

In the absorbent body according to another embodiment, at least one of the first elastic member and the second elastic member may have elasticity of a load of 0.1 to 10 N/25 mm when 50% elongation is performed at a speed of 300 mm/minute.

In the absorbent body according to another embodiment, at least one of the first delimiting part and the second delimiting part may include the absorbing member in a region of at least a portion of the first delimiting part and the second delimiting part.

In the absorbent body according to another embodiment, at least one of the first delimiting part and the second delimiting part may include a welded portion.

In the absorbent body according to another embodiment, volume of the housing part may be larger than volume of the absorbing member.

An absorbent article according to an embodiment of the present invention includes the absorbent body described above.

The absorbent article according to an embodiment can follow various postures of a wearer.

A method of manufacturing an absorbent body according to an embodiment of the present invention includes the steps of laminating an absorbing member on a first elastic member, providing a second elastic member on the first elastic member and on an absorbing member laminated on the first elastic member, and forming a plurality of housing parts after providing the second elastic member, and in the method, the plurality of housing parts are delimited from each other by a first delimiting part along a first direction and a second delimiting part along a second direction intersecting the first direction, and house the absorbing member.

According to the method of manufacturing an absorbent body according to an embodiment, since the absorbing member is sandwiched between the first elastic member and the second elastic member, the absorbent body manufactured can have elasticity in a plurality of directions. The absorbent body includes the plurality of housing parts, and the plurality of housing parts are delimited from each other by the first delimiting part along the first direction and the second delimiting part along the second direction intersecting the first direction. In this manner, since the housing parts are delimited in directions intersecting each other, the housing parts can hold the absorbing member at a predetermined position without fixing the absorbing member by an adhesive. In this manner, since no adhesive for fixing the absorbing member may be used, the first and second elastic members can have elasticity in a plurality of directions in a state in which loss of elasticity is suppressed.

In the method of manufacturing an absorbent body according to another embodiment, at least one of the first delimiting part and the second delimiting part may include the absorbing member in a region of at least a portion of the first delimiting part and the second delimiting part.

In the method of manufacturing an absorbent body according to another embodiment, at least one of the first delimiting part and the second delimiting part may include a welded portion, and the welded portion may be formed by welding the first elastic member and the second elastic member by a thermal welding method or an ultrasonic welding method.

1. An absorbent body comprising:
a first elastic member;
a second elastic member; and
an absorbing member laminated between the first elastic member and the second elastic member,
wherein the first elastic member forms, together with the second elastic member, a plurality of housing parts, and
the plurality of housing parts are delimited from each other by a first delimiting part along a first direction and a second delimiting part along a second direction intersecting the first direction, and house the absorbing member.

2. The absorbent body according to claim 1, wherein the absorbing member includes a water absorbing polymer.

3. The absorbent body according to claim 1, wherein at least one of the first elastic member and the second elastic member has liquid permeability.

4. The absorbent body according to claim 1, wherein at least one of the first elastic member and the second elastic member includes non-woven fabric.

5. The absorbent body according to claim 1, wherein at least one of the first elastic member and the second elastic member has elasticity of a load of 0.1 to 10 N/25 mm when 50% elongation is performed at a speed of 300 mm/minute.

6. The absorbent body according to claim 1, wherein at least one of the first delimiting part and the second delimiting part includes the absorbing member in a region of at least a portion of the first delimiting part and the second delimiting part.

7. The absorbent body according to claim 1, wherein at least one of the first delimiting part and the second delimiting part includes a welded portion.

8. The absorbent body according to claim 1, wherein volume of the housing part is larger than volume of the absorbing member.

9. An absorbent article including the absorbent body according to claim 1.

10. A method of manufacturing an absorbent body, the method comprising the steps of:
laminating an absorbing member on a first elastic member;
providing a second elastic member on the first elastic member and on an absorbing member laminated on the first elastic member; and
forming a plurality of housing parts after providing the second elastic member,
wherein the plurality of housing parts are delimited from each other by a first delimiting part along a first direction and a second delimiting part along a second direction intersecting the first direction, and house the absorbing member.

11. The method of manufacturing an absorbent body according to claim 10, wherein at least one of the first delimiting part and the second delimiting part includes the absorbing member in a region of at least a portion of the first delimiting part and the second delimiting part.

12. The method of manufacturing an absorbent body according to claim 10, wherein at least one of the first delimiting part and the second delimiting part includes a welded portion, and the welded portion is formed by welding the first elastic member and the second elastic member by a thermal welding method or an ultrasonic welding method.
